(19) United States
(12) Patent Application Publication
(10) Pub. No.: US 2026/0167997 A1
(43) Pub. Date: Jun. 18, 2026

(54) CONVERSION OF LYSINE PHOSPHATE TO 1.5 PENTANEDIAMINE AND SEPARATION OF DIAMINES FROM AMINO AND CARBOXYLIC ACIDS

(71) Applicant: ARCHER-DANIELS-MIDLAND COMPANY, Decatur, IL (US)

(72) Inventors: Karen Brown, Decatur, IL (US); Erik Hagberg, Decatur, IL (US); Padmesh Venkitasubramanian, Forsyth, IL (US); John Soper, Mt. Zion, IL (US); Pam Anklam, Lovington, IL (US); Shimin Mao, Decatur, IL (US)

(21) Appl. No.: 18/730,485

(22) PCT Filed: Jan. 18, 2023
(86) PCT No.: PCT/US2023/060820

§ 371 Date: Jul. 19, 2024

Related U.S. Application Data
(60) Provisional application No. 63/301,481, filed on Jan. 20, 2022.

(51) Int. Cl. C12P 13/00; B01D 15/18; B01D 15/20; B01D 15/36; B01D 15/42; C07C 209/86

(52) U.S. Cl. CPC C12P 13/001; B01D 15/1835; B01D 15/203; B01D 15/363; B01D 15/424; C07C 209/86

(57) ABSTRACT

A method of making 1,5 pentanediamine (a.k.a. PDA, pentamethyldiamine, PMDA and cadaverine) by decarboxylation of lysine using lysine decarboxylase in the presence of sulfate, or more preferably phosphate anion salts of lysine is described. The process works at high pHs at which the decarboxylase does not function using HCL salts of lysine and at substrate concentrations not achievable with lysine HCL at the ordinary pH optima of the carboxylase. In addition, a chromatographic process for purifying the PDA from lysine and the conjugate anions of the lysine salt is described that is applicable as a general process for purifying any diamine from a product mixture comprising amino and/or carboxylic acids along with their conjugate anions, which method uses a strong anion exchange resin as the stationary phase. The method does not utilize any organic solvent and is shown to be economical and efficient for industrial scale isolation of diamines from complex mixtures containing amino and/or carboxylic acids and conjugate anions from the amine salts.

### FIELD OF INVENTION

The present disclosure relates to processes for separating a diamine from a mixture comprising the diamine and one or more amino and/or carboxylic acids in high yield at low coast without the use of any organic solvent.

### BACKGROUND

Diamines are used for the synthesis of polyamides and polyurethanes. For example, 1,4-diaminobutane and 1,6-diaminohexane are used for the synthesis of nylon-4,6 and -6,6, respectively. The diamines are produced by biochemical or chemical methods. The biochemical method utilizes culturing a transformed microorganism engineered to secrete either a diamine to the culture medium or an amino acid decarboxylase to a culture medium containing the amino acid (see for example U.S. Pat. Nos. 11,124,812, 11,053,524, 11,053,525, 10,626,425, 10,870,871, 10,711,289, 10,640,798, 10,472,636, 10,150,977, 9,745,608, 9,644,220, 9,365,876, 9,115,362, 8,741,623, each of which is incorporated herein by reference in its entirety). In the case of microorganisms secreting amino acid decarboxylase (see for example U.S. Pat. No. 8,871,477, incorporated herein by reference in its entirety), an amino acid such as lysine or ornithine is added to the culture medium clarified of the biomass to produce the diamine by action of the decarboxylase. Some methods utilize diammonium salts of carboxylic acids to produce the diamines (see for example U.S. Pat. No. 8,742,060, incorporated herein by reference in its entirety). In any of the above-mentioned methods, the crude product is a complex mixture containing amino and/or carboxylic acid(s) from the amino and/or carboxylic acid used to produce the diamine and the culture media usually contain one or more carboxylic acid metabolites such as acetic acid, oxalic acid, succinic acid, tartaric acid, lactic acid and/or salts thereof, as well as the conjugate ions of amine or carboxylate functional groups such as phosphate, sulfate, HCL, chloride, ammonium, sodium, potassium and the like, which may also be present due the their addition as mineral nutrients in a culture medium. Thus, the separation of the diamine efficiently high yield and purity as well as low cost from such a complex mixture continues to be a challenging process.

Several methods have been reported to isolate and purify diamine such as 1,5-pentanediamine from reaction mixtures. JP581215 (B2) and JP2016033138 (A), each of which is incorporated herein by reference in its entirety, disclose distillation method to isolate 1,5-pentanediamine from a mixture obtained from a reaction mixture of thermal decomposition or enzymatic conversion. Several methods have been reported to isolate 1,5-pentanediamine from a reaction mixture by adsorbing the diamine on neutral or anionic resins followed by eluting the diamine from the resin, see for example CN109942437 (A), CN110563594 (A), CN110143882 (A), CN108276293 (A), U.S. Pat. Nos. 9,878,321 (B2), 10,576,467 (B2), 9,617,202 (B2), Howel and Byus, Analytical Biochemistry (2002) 311 (2), 127-32], and Lin et al. Chinese Journal of Chromatography (2018) 36 (11), 1189-1193; each of which is incorporated herein by reference in its entirety. U.S. Pat. Nos. 10,265,642 (B2) and 10,343,084 (B2), each of which is incorporated herein by reference in its entirety, disclose a method of separating of at least one amine, chosen from diamines and omega-amino acids, from a feed mixture using simulated moving bed (SMB) adsorptive technology. They further disclose that useful adsorbents are activated carbon, floridin, diatomite, molecular sieves, alumina, silica, silica-alumina, titania, polymeric resins containing one or more groups selected from sulfonate, hydroxy, amino, halogen, pyridyl, mono-substituted amino, disubstituted amino, acyl, acyloxy, keto, alkoxy, and polymeric resins containing immobilized silver or lead, commonly known as immobilized metal affinity columns. Other cited adsorbents are Orpheus silica-based stationary phase adsorbent, and Amberlite XAD-4, XAD-7, XAD-8 and XAD-418 resins, non-polar resins. Elution solvents can be chosen from water, diols, esters, nitriles, ketones, ethers, methanol, diols, esters, and aliphatic and cyclic ethers such as dimethyl ether, tetrahydrofuran and dioxane.

Even though all the above cited methods have had some success, they have been limited either by costly material, byproduct-waist generation, suboptimal yield, or combination thereof. Thus, a need exists for a simple and more efficient method for separating diamines from a reaction mixture or fermentation broth which contains amino and/or carboxylic acid with optimal diamines yield at a lower coast and without the need to use organic solvents.

### SUMMARY OF THE INVENTION

A first aspect pf the of the invention is a method of forming 1,5-pentanediamine (PDA) comprising contacting a mixture containing at least a 0.3M solution of a lysine salt with sulfate or phosphate as the conjugate anion with an enzyme having a lysine decarboxylase activity for a time sufficient to convert at least 50% of the lysine to PDA. In exemplary embodiments, the lysine decarboxylase may be encoded by the cadA gene of E. coli.

In preferred embodiments the conjugate anion is phosphate. In preferred embodiments the mixture is at a pH of 8.0 to 9.0. In more preferred embodiments the mixture contains 0.5 to 2.9 M lysine phosphate at a pH of 8.0 to 9.0. In most preferred embodiments the pH is 8.0 to 8.5. In best practices of the forgoing embodiments, at least 90% of the lysine is converted to PDA. In exemplary examples the lysine salt is formed by adding sulfuric acid, or more preferably phosphoric acid to lysine free base in sufficient quantities to adjust the mixture to the desired pH.

The forgoing embodiments may further include separating the PDA from lysine and the conjugate anion by contacting the reaction mixture with a strong base ion exchange resin and eluting the PDA from the resin, wherein the eluted PDA is at least 85% pure containing less than 15% of the lysine or conjugate anion. In preferred embodiments the strong base ion exchange resin is configured in a simulated moving bed apparatus.

A second aspect of the invention is directed to a process for separating a diamine from an aqueous feed stock containing the diamine, and at least one salt of an amino acid or carboxylic acid, comprising: contacting the aqueous feed stock at a pH in the range 5.0 to 10.0 with a bed of a strong anion exchange resin, wherein the diamine is eluted from the resin in fraction that has a purity of at least 85% and contains less than 10% (w/w) of the amino acid or carboxylic acid.

In some embodiments, the diamine is selected from 1,2-ethylenediamine, 1,3-propanediamine, 1,4-butanediamine (putrescine), 1,5-pentanediamine (cadaverine), 1,6-hexanediamine, 1,7-heptaindiamine, and the like.

In one embodiment, the diamine is 1,5-pentanediamine (PDA) and the at least one amino acid salt is a lysine salt.

In one embodiment, the feed stock is obtained by decarboxylation of lysine by the action of lysine decarboxylase.

In another embodiment, the lysine salt is selected from the group consisting of lysine phosphate and lysine sulfate.

In another embodiment, less than 10% of the sulfate or phosphate conjugate anion of the lysine salt is present in the eluted fraction containing the diamine.

In another preferred embodiment, the lysine salt is lysine phosphate.

In some other embodiments, the strong anion exchange resin comprises a quaternary ammonium salt.

In another more preferred embodiment, the purity of PDA in the eluted fraction is at least 95% and contains less than 5% (w/v) lysine.

In other embodiments, the at least one amino and/or carboxylic acid is eluted from the anion exchange bed with a hydroxide salt.

In some embodiments, the hydroxide salt is ammonium hydroxide.

In another embodiment, the hydroxide solution is sodium hydroxide or potassium hydroxide.

In other embodiments, the feed stock has a pH in the range of 8.0 to 9.0.

In other embodiments, the bed is packed into a set of columns contained in a simulated moving bed apparatus configured with a feedstock loading zone, a raffinate elution zone and a regeneration zone, wherein: (a) the feedstock is loaded onto a first column segment defining the feedstock loading zone, (b) the 1,5-pentanediamine is eluted from a second column segment downstream from the feedstock loading zone relative to the direction of flow of liquid through the column segments, (c) lysine and the conjugate anion of the salt of the lysine are eluted from a third column segment in the raffinate elution zone, and (d) the column is regenerated in the regeneration zone; and where the raffinate elution zone is not in fluid communication with the feedstock loading zone and the regeneration zone is not in fluid communication with the raffinate elution zone.

In a preferred embodiment, a water rinse is introduced into the column in a segment upstream of the first column segment defining the feedstock loading zone; a hydroxide salt solution is introduced into the column in a segment within the raffinate elution zone; and water is introduced into a column segment in the regeneration zone.

In another embodiment, the lysine salt is lysine phosphate.

In other preferred embodiments, the simulated moving bed apparatus comprises 12 column segments of which, the feedstock loading zone comprises 5 column segments, the raffinate elution zone comprises 3 column segments and the regeneration zone comprises 4 column segments.

In a preferred embodiment, the lysine salt is lysine phosphate.

A second aspect of the invention is directed to a method of producing a diamine. In one embodiment, the method comprises:
(a) culturing a host microorganism engineered to produce a diamine in a culture medium comprising a source of phosphate or sulfate to form a phosphate or sulfate salt;
(b) processing the culture of the microorganism to produce a feed stock comprising the diamine;
(c) contacting the feed stock with a bed of a strong anion exchange resin in a column, and
(e) eluting the diamine from the column in a first fraction separate from a second fraction containing amino and carboxylic acid salts;
wherein the diamine is eluted from the column with at least 95% purity and contains less than 5% amino and/or carboxylic acid.

In some embodiments, the diamine is selected from 1,3-propane diamine, 1,4-butanediamine (putrescine), 1,5-pentanediamine (cadaverine), 1,6-hexanediamine, and 1,7-heptaindiamine.

In some preferred embodiments, the column comprises a set of columns contained in a simulated moving bed apparatus configured with a feedstock loading zone, a raffinate elution zone and a regeneration zone, wherein: (a) the feedstock is loaded onto a first column segment defining the feedstock loading zone, (b) the diamine is eluted from a second column segment downstream from the feedstock loading zone relative to the direction of flow of liquid through the column segments, (c) lysine and the conjugate anion of the salt of the lysine are eluted from a third column segment in the raffinate elution zone, and (d) the column is regenerated in the regeneration zone; and where the raffinate elution zone is not in fluid communication with the feedstock loading zone and the regeneration zone is not in fluid communication with the raffinate elution zone.

In another embodiment, the method is directed to a method of producing 1,5-pentanediamine (PDA) comprising:
(a) obtaining a culture broth resulted from fermenting a host microorganism engineered to produce lysine;
(b) removing the microorganism from the culture broth;
(c) adding a source of phosphate or sulfate to the culture broth to form a phosphate or sulfate salt of lysine;
(d) decarboxylating the lysine by contacting culture broth containing the sulfate or phosphate with lysine decarboxylase forming a culture broth containing the lysine salt and PDA;
(e) contacting the culture broth containing the lysine salt and PDA with a bed of a strong anion exchange resin in a column, and
(f) eluting the PDA from the column in a first fraction separate from a second fraction containing lysine and lysine salt;
wherein the PDA is eluted from the column with at least 95% purity and contains less than 5% lysine.

In a preferred embodiment, the lysine salt is lysine phosphate.

In a more preferred embodiment, the column comprises a set of columns contained in a simulated moving bed apparatus configured with a feedstock loading zone, a raffinate elution zone and a regeneration zone, wherein: (a) the feedstock is loaded onto a first column segment defining the feedstock loading zone, (b) the 1,5-pentanediamine is eluted from a second column segment downstream from the feedstock loading zone relative to the direction of flow of liquid through the column segments, (c) lysine and the conjugate anion of the salt of the lysine are eluted from a third column segment in the raffinate elution zone, and (d) the column is regenerated in the regeneration zone; and where the raffinate elution zone is not in fluid communication with the feedstock loading zone and the regeneration zone is not in fluid communication with the raffinate elution zone.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1A shows a pulse test comparison of eluting 1,5-pentanediamine (cadaverine) from DOW 22™, PA208™ and HPA 25 L™ using deionized water as effluent.

FIG. 2B shows a pulse test comparing the effect of pH on the retention of 1,5-pentanediamine from DOW 22 ™, PA208 ™ and HPA 25 L™.

FIG. 2 shows a pulse test showing the elution profile and purity of 1,5-pentanediamine from a column packed with DOWEX 22™ anion exchange resin.

FIG. 3 shows a pulse test showing the elution profile and purity of 1,5-pentanediamine from a column packed with PA308™ anion exchange resin.

FIG. 4 shows a pulse test showing the elution profile and purity of 1,5-pentanediamine from a column packed with HPA25L™ anion exchange resin.

FIG. 5 shows a column elution profile for separating 1,5-pentanediamine from phosphate and lysine over a PA 209 strong base ion resin.

FIG. 6 shows a schematic representation of a simulated moving bed chromatographic system for the purification of diamine from a complex reaction mixture.

FIG. 7A shows for comparative purposes, the conversion of lysine to 1,5-pentanediamine (a.k.a PDA or cadaverine) by E. coli lysine decarboxylase over time using lysine HCl as the lysine salt. FIG. 7B shows a time course for the conversion of lysine using lysine sulfate as the lysine salt according to one embodiment of the invention. FIG. 7C shows a time course for the conversion of lysine using lysine phosphate as the lysine salt according to another embodiment of the invention.

FIG. 8 shows the percent conversion of lysine to 1,5-pentanediamine at various pH values using a 1 M solution of lysine free base adjusted to the indicated pH with phosphoric acid.

### DETAILED DESCRIPTION

Several aspects of the invention are described herein with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events, unless otherwise specifically indicated. Furthermore, not all illustrated acts or events are required to implement a methodology in accordance with the features described herein.

The terminology used herein is for the purpose of describing exemplary embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

In this disclosure the term “about” or “approximately” means a range of up to 10%, preferably 5%, and mor preferably 3% of a given value. In this disclosure the term “substantially” refers to something that can be done to a great extent or degree.

As used herein, the term “diamine” refers to any organic compound containing two or more amino groups. Example of the diamines of the invention are aliphatic diamine such as, but not limited to 1,2-ethylenediamine (also known as diaminoethane), 1,3-propanediamine (also known as trimethylenediamine), 1,4-butanediamine (also known as putrescine or tetramethylenediamine), 1,5-pentanediamine known (also as cadaverine or pentamethylenediamine, PDA, or PMDA), 1,6-hexanediamine (also known as hexamethylene diamine), 1,7-heptain diamine (also known as heptamethylenediamine) and isomers, derivatives, and analogs thereof, and aromatic amines such as, but not limited to o-, m-, or p-phenylene diamine, 4,4′-diaminobiphenyl and isomers, derivatives, and analogs thereof. It should be noted that some diamines are known by several acceptable names in the art such as, but not limited to those listed above.

As used herein, the term “fermentation” refers to a process of production of a compound such as a diamine, an organic acid, an amino acid or an enzyme such as lysine decarboxylase by growing a wild-type or an engineered microorganism that produces the compound or enzyme of interest.

As used herein, the term “fermentation broth” refers to a liquid medium in which a microorganism converts organic carbon sources to a desired organic material such as but not limited to a diamine, an amino acid, or amino acid decarboxylase; and may contain other organic materials such as carboxylic acid and alcohols as by products, and typically also includes nutrients and salts required for organism growth, pH control or to form salts of compounds produced by the fermentation. The term includes whole broth containing the microorganism of interest and clarified broth when the microorganism is separated from the remaining components.

As used herein, the term “carbon source” refers to carbon containing nutrients required for growing a microorganism and producing a desired product. Carbon sources may include carbohydrates such as but not limited to glucose, fructose, sucrose, and starch; tryptone, carboxylic acids and/or salts thereof such as but not limited to acetic acid, tartaric acid, citric acid, and the like; amino acids and/or salts thereof; and triglycerides.

The terms “bed volume” or “column volume” refers to the liquid volume within a packed column or bed.

A first aspect of the present disclosure is the discovery that lysine sulfate and lysine phosphate, and particularly lysine phosphate are superior to lysine HCl as substrates for conversion of lysine to PDA using lysine decarboxylase in terms of achieving high conversion rates at lower enzyme dosages. In addition, lysine sulfate and lysine phosphate can be used at much higher concentrations than HCl making the reaction with these substrates more suitable for commercial scale production. Moreover, use of lysine sulfate or lysine phosphate allows reactions with lysine decarboxylase to proceed at broad pH ranges from 5.0 to 9.0 with high efficiency.

Enzymatic conversion of lysine to PDA by lysine decarboxylase designated CadA, which is encoded by the cadA gene from E. coli, is a known reaction. CadA is a pyridoxal 5 phosphate dependent lysine decarboxylase that is induced by lysine in acidic systems. The native useful pH range of the enzyme is between 5-7, with a recorded pH optimum of 5.5. If the pH is increased beyond 7, the enzyme is known to disassociate and form low activity dimers (Kou et al. Characterization of a new lysine decarboxylase from Aliivibrio salmonicida for cadaverine production at alkaline pH, Journal of Molecular Catalysis B: Enzymatic, vol 133, Supp. 1, 588-594, 2016). Literature shows a steep reduction in activity at pHs beyond 5.5 (Kou et al 2016). Lysine free base has a pH of 10.2, which is far too high for CadA to work, therefore a an acidic (anionic) salt of lysine must be used to lower the pH. However, prior studies performed with CadA for lysine decarboxylation have exclusively used lysine hydrochloride as a substrate, and some have used hydrochloric acid to maintain pH during the reaction. Alternative lysine salts such as lysine sulfate or lysine phosphate have not been evaluated.

The present disclosure reveals high conversion of lysine to PDA using a lysine decarboxylase, exemplified by CadA, can occur when lysine sulfate and especially when lysine phosphate is used at concentrations greater than 0.5M up to the solubility limit of lysine phosphate of about 3.0 M. In contrast, the use of lysine HCl is limited by its solubility at higher concentrations. A 2.75M lysine HCL solution pH adjusted to 5.5 is insoluble at temperatures where CadA is active. Use of lysine phosphate or lysine sulfate increases solubility and allows the reaction to progress at the CadA temperature optimal of 37° C. with much higher conversion rates than possible with lysine HCl. Moreover, use of these salts, especially lysine phosphate, can extend the pH range of the CadA enzyme to as high as 9.0, which ordinarily would cause disassociation of the enzyme when lysine HCL is used.

To evaluate the effects of conjugate anion salts of lysine on the conversion of lysine to PDA using lysine decarboxylase, a strain of E. coli designated BB16.9.8 was engineered to over express CadA. BB16.9.8 contains a copy of the T7 polymerase under control of the lac promoter, which is inducible by lactose or IPTG. A copy of the E. coli lysine decarboxylase cadA gene under control of the T7 promoter was also integrated into the genome adjacent to the thrC gene. The strain was grown into log phase in the presence of inducible amounts of lactose to overexpress CadA, then quickly chilled and centrifuged to obtain a cell paste. The cell paste was lysed with using a BUG BUSTER™ cell chemical lysis media which makes the cells porous, to form a crude homogenate, which was centrifuged to obtain a clarified crude extract having a protein concentration of about 4.0 mg/ml designated herein as CadA lysate. One molar solutions of lysine HCl, lysine sulfate and lysine phosphate were prepared by adding hydrochloric acid, sulfuric acid or phosphoric acid in molar equivalents to a 1 M solution of lysine free base in amounts sufficient to adjust the pH to a desired level. These samples were evaluated for the ability of the CadA lysate to convert lysine to PDA using an assay mixture that contained pyridoxal phosphate which is a required cofactor for lysine decarboxylase. Exemplary reactions were performed with 10 ml to 30 liters of the lysine salt solutions, and the amount of CadA lysate added was varied in different experiments.

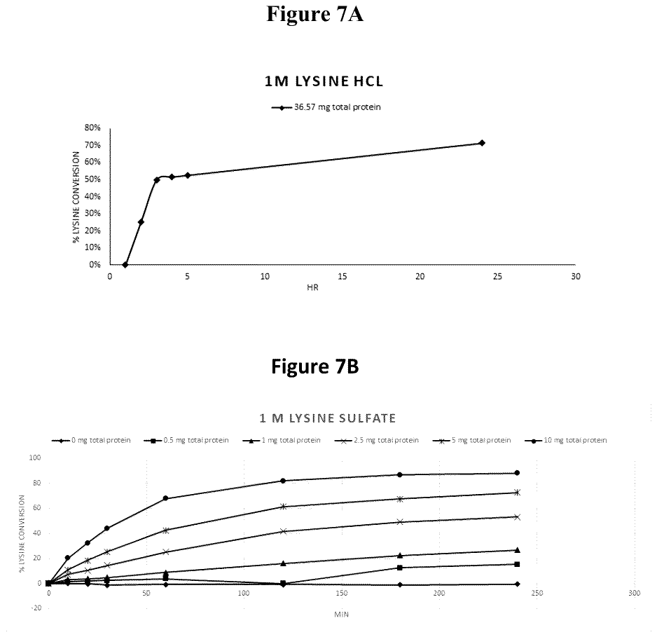

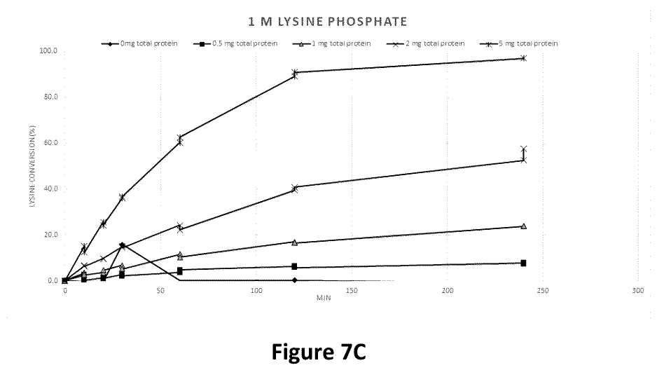

In a first illustrative experiment with 250 g of 1M lysine HCl, pH 5.5, 37 mg of the CadA lysate was incubated in with that amount of lysine HCL for period of 24 h with time points taken each hour for the first 5 hours and at 24 hours. The reactions at selected time points were halted by heating at 70° C. for one hour. The percent conversion of lysine to PDA was assessed by HPLC. FIG. 7A shows the reaction proceeded linearly for the first 3 hours by which time approximately 50% of the lysine was converted to PDA and slowed thereafter so that at 24 hours only about 70% conversion was obtained.

In other experiments using 1M lysine sulfate and 1M lysine phosphate adjusted to pH 5.5, differing amounts of CadA (measured by total protein) were added to the reaction mixture and time points were taken over a much shorter reaction period of only 4 hours because the reaction with these salts had previously been shown by the inventors to be much faster than with lysine HCl. FIG. 7B shows that for lysine sulfate, with only 5 mg of crude extract protein in the 10 ml reaction, about 60% conversion to PDA occurred after two hours and at 6 hours the conversion reached about 70%. FIG. 7C shows a yet more surprising result, that with lysine phosphate and only 5 mg of protein, just over 90% of the lysine was converted to PDA after two hours and 6 hours there was over 95% conversion.

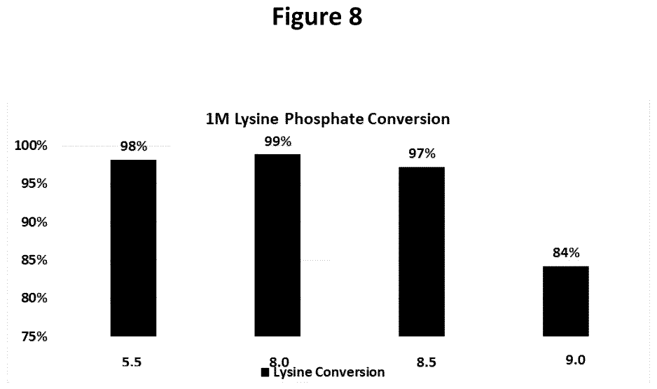

Another benefit of using lysine sulfate or more preferably, lysine phosphate for the conversion of lysine to PDA by lysine decarboxylase is a broadening of the useful pH range of the enzyme. As mentioned herein above, the E. coli CadA lysine decarboxylase has a pH optimum of 5.5 and rapidly loses activity at pHs greater than 7.0. In contrast, the inventors discovered that use of lysine sulfate or lysine phosphate, and particularly lysine phosphate allows the enzyme to retain activity at least up to pH 9.0. Most methods of producing lysine by fermentation produce lysine in the free base form, which has a pH of about 10.2, well above useful pH range of the E. coli lysine decarboxylase. Using a sulfate or more preferably a phosphate salt of lysine broadens the useful pH range of the enzyme, which in turn allows for use of less of the acid (sulfuric or phosphoric acid) that provides the conjugate anion for the lysine salt than would be required for forming lysine HCL at a pH in a range where the CadA enzyme is active, thereby reducing the amount of anion that must be separated from the PDA. FIG. 8 shows that between pH 5.5 and 8.0 at least 84% of the lysine in a 1M solution of lysine phosphate could be converted to PDA in 24 hours using only 5 mg of CadA lysate in a 10 ml reaction. At least 97% conversion was observed at pH's between 5.5 and 8.5.

Another benefit of using lysine sulfate, or most preferably lysine phosphate is the ability achieve much high concentrations than achievable with lysine HCl. Examples 1 through 9 shows high conversion rates after 24-hour reaction using CadA with reactions containing 0.57 to 2.86 M lysine adjusted with phosphoric acid to at a variety of pH values. Accordingly, another feature of the invention is converting lysine to PDA in a reaction containing lysine decarboxylase and at least 0.3 M lysine adjusted to a pH of 8.0 to 9.0 forming lysine phosphate. In exemplary embodiments, the mixture contains 0.5 M to 2.9 M lysine adjusted to a pH of 8.0 to 9.0. More preferably the lysine concentration is 1.0 M to 2.5 M. or more preferably 1.5 M to 2.25 M and the pH is 8.0 to 8.5. In most preferred embodiments the lysine concentration is 1.75 to 2.25 M and the pH is 8.5. Reaction times and enzyme amounts can be varied to achieve at least 50%, at least 70%, at least 80% at least 90% and most preferably at least 95% conversion of lysine to PDA. In some reactions at least 98% conversion may be obtained.

A second aspect of the invention is directed to a process for separating PDA from lysine and the conjugate anion of the lysine salt. The invention is applicable to separating any diamine from an aqueous feed stock containing the diamine, and at least one salt of an amino acid and/or carboxylic acid. This aspect of the invention comprises: contacting an aqueous feed stock at a pH in the range 5.0 to 10.0, preferably 6.0 to 9.5, more preferably 6.5 to 9.0, more preferably 7.0 to 9.0, more preferably 8.0 to 9.0, and most preferably about 8.5 with a bed of a strong anion exchange resin, wherein the diamine is eluted first from the resin having at least 85%, 90%, 92%, 94%, 95%, 96%, 97%, 98%, or 99% (w/w) purity and containing less than 10%, 8%, 6%, 5%, 4%, 3%, 2%, or 1% (w/w) of the amino acid or carboxylic acid and the amino and/or carboxylic acid salt and/or conjugate ions of the amino acid salt are eluted separately by a hydroxide salt solution. The feed stock is an aqueous solution, typically a clarified fermentation broth comprising at least a diamine product and one or more of the reagents that reacted to produce the diamine such as, but not limited to unreacted starting material(s) organic and inorganic salts such as amino and carboxylic acids and salts thereof, dicarboxylic acids and salts thereof, tricarboxylic acid and salts thereof, pyridoxal 5′-phosphate, inorganic salts such as, but not limited to alkali and alkaline earth metals salts of phosphate, sulfate, chloride and the like, as well as other metabolites such as, but not limited to ethanol, propanol and the like found in a culture medium, and byproducts produced by any chemical or biochemical process to produce the diamine. The feed stock may contain alcohol such as ethanol or propanol of less than 10%, preferably 8%, preferably 6%, preferably 5% (w/w) produced by the fermentation process. Amino acids and/or carboxylic acids are eluted from the resin with an aqueous solution of hydroxide salt such as, but not limited to ammonium hydroxide, sodium hydroxide, potassium hydroxide, and the like at a concentration in in the range of 0.5% (w/v) to 10% (w/v), preferably 1% (w/v) to 9% (w/v), more preferably 2% (w/v) to 8% (w/v), more preferably 3% (w/v) to 7% (w/v), and most preferably 4% (w/v) to 6% (w/v).

The process of the invention is particularly suitable for separating any diamine from a feed stock of a reaction mixture or fermentation broth comprising amino and/or carboxylic acids. In some preferred embodiments, the diamine is selected from the group consisting of 1,2-ethylenediamine, 1,3-propanediamine, 1,4-butanediamine (putrescine), 1,5-pentanediamine (cadaverine), 1,6-hexanediamine, and 1,7-heptainediamine. In one preferred embodiment, the diamine is 1,5-pentanediamine. One of the many advantages of this method is that the diamine is the early eluting fraction from the resin leading to substantial improvement in recovering the product and the late eluting product(s) such as the amino acid salt, conjugate ions of the amino acid salt and other inorganic salts may be recovered and recycled to a fermentation process to produce the diamine or converted to other useful products.

Strong anion exchange resins a are typically a polymeric matrix that contains quaternary ammonium groups. Standard commercially available strong anion exchange resins contain either —N⁺(CH₃)₃ (type 1 resins) or —N⁺(CH₃)₂ (C₂H₄OH) (type 2 resins). The separation of chemical compounds on ion exchange resins is deponent on the degree of interaction between charge groups of the stationary phase and molecules in the mobile phase. For example, a strong ion exchange resin comprising quaternary ammonium cations would strongly interact with and preferentially retain negatively charged molecules, whereas neutral and positively charged molecules would interact less readily with the resin and more preferentially flow with 5 the mobile phase. Examples of commercially available strong anion exchange resins and their manufacturer are listed in Table 1. In some preferred embodiments, the anion exchange resin is selected from DOW 22™, HPA 25L™, and Mitsubishi PA DIAION PA308™. It is a porous resin containing the trimethylammonium cation and efficiently separate diamines from a mixture of diamines and salts amino and/or carboxylic acids at a high flow rate.

TABLE 1

| Manufacturer | Resin |
|---|---|
| MITSUBISHI ™ | SA10A, SA11A, SA12A, NSA100, PA306, PA |
| | 308, PA312, PA316, HPA25, UBA100, UBA120, |
| | SA20A, SA21A, PA408, PA412, PA416, PA418, |
| | PAF418, UBA200. |
| PUROLITE ™ | A200, A300, A400, A500, NRW5010, NRW5070, |
| | A501P, MPR1000, A500TLPLUS, SGA550, |
| | A300E, A420S, A502PS, A503S, A860, A860S, |
| | PPA503, PPA502PS, PPA860S, SSTA63, SSTA64. |
| LEWATIT ™ | S 5128, S 5528, S 6268, S 6368, S 7468, K 6362, |
| | K 6367, K 6462, K 7333, A 8071, TP 106, TP 108, |
| | M500 MonoPlus, M 800 MonoPlus. |
| DuPONT ™ | Marathon A, IRA402, IRA458, IRA900, HPR550, |
| | HPR4100, HPR 4200, HPR4700, HPR4800, |
| | HPR9100, Amberlite FPA22, IRA910, MSA-1, |
| | A-26. |
| FINEX ™ | AS 541M. |
| TULSION ™ | A-23P, A-32, A-62, A-72, A-36MP, A-62MP, |
| | A-72MP. |
| RESINEX ™ | TPS-2300, TPS-2308, TPS-2320, TPS-2400, |
| | TPS-2420, TPS-2425, TPS-2200, TPS-2207 |
| CARB-CHEM ™ | AN-22, AN-23, AN-24. |
| INDION ™ | 810, 820, 830, FFIP, GS 300, GS 400, NSSR, NIP. |
| GENERAL- | A302, A304, A307, A351, A352, D890, A313 |
| TECHNOLOGY ™ | |

As indicated above, the mixture applied to the resin may be obtained from any chemical or biochemical process that produces the diamine or an amino acid to be converted to the diamine. In preferred embodiments, the feed stock is a clarified fermentation broth obtained from fermenting microorganisms to produces the desired diamine or amino acid. In embodiments illustrated by example herein, a clarified fermentation broth is obtained by fermenting a bacteria to produce lysine is and removing the bacteria by filtration to produce clarified fermentation broth containing lysine fee base. A mineral acid, illustrated by sulfuric acid or phosphoric acid or mineral salt of phosphate or sulfate is added to the clarified broth to form the lysine sulfate and lower the pH to where lysine decarboxylase is active. The mixture is pH adjusted to be in the range that is optimal for functioning of lysine decarboxylase, which is added to the mixture for a time sufficient to convert the lysine to 1,5 pentanediamine (PDA). At the end of the reaction time, the mixture is applied to a strong anion ion exchange resin in a column, which is eluted to obtain a first fraction that is at least 85%, more preferably at least 90% and still more preferably at least 95% PDA, and containing less than 15%, less than 10% or more preferably less than 5% of lysine plus the conjugate sulfate or phosphate anion, followed by elution of the column with a hydroxide base to remove residual lysine and the conjugate anions from the resin.

The invention may be practiced with fermentation of microorganisms known in the prior art to produce any diamines. Examples of microorganisms producing diamines: U.S. Pat. No. 10,711,289, incorporated herein by reference in its entirety, discloses engineered microorganisms to produce ethylenediamine, 1,3-propanediamine, 1,4-butanediamine, 1,5-pentanediamine, 1,6-hexanediamine, and 1,7-heptaindiamine. U.S. Pat. No. 10,472,636, incorporated herein by reference in its entirety, discloses microorganisms engineered to produce 1,3-propanediamine and 1,5-pentanediamine (cadaverine). U.S. Pat. Nos. 11,124,812, 11,053,525, 11,053,524, and 10,870,871, each of which is incorporated herein by reference in its entirety, disclose one or more microorganisms such as Corynebacterium glutamicum, Providencia rettgeri, Brevibacterium flavum, Brevibacterium lactofermentum, and Serratia marcescens, and Escherichia coli engineered to produce 1,4-butanediamine (putrescine). U.S. Pat. Nos. 8,741,623, 10,626,425; 10,640,798, 9,745,608, 9,644,220, 9,365,876, 9,115,362, and 8,741,623, each of which is incorporated herein by reference, discloses one or more microorganisms engineered to produce 1,5-pentanediamine. Among the disclosed microorganisms to produce of 1,5-pentanediamine are Corynebacterium glutamicum, Providencia rettgeri, Brevibacterium flavum, Brevibacterium lactofermentum, and Serratia marcescens, and Escherichia coli. U.S. Pat. No. 10,150,977, incorporated herein by reference in its entirety, discloses engineered Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida to produce 1,6-hexanediamine.

The mixture applied to the column may be obtained by filtering any solids and/or removing the microorganism from the broth to form a clarified fermentation broth. Also, in some instances, the cells of the microorganism may be lysed, and the solids removed by filtration to produce the feed stock for the column or for a reaction that will form the diamine that will be applied over the column. Also, in some instances, it may be desired to remove proteins and nucleic acids from the feed stock prior to isolating the diamine by well-known methods in the art such as but not limited to acidifying the culture broth to precipitate proteins and nucleic acids.

In some embodiments, the feed stock may be prepared from a culture medium containing a diamino acid such as lysine or ornithine using a microorganism engineered to express an enzyme with an amino acid decarboxylase activity. For example, U.S. Pat. No. 8,871,477, incorporated herein by reference in its entirety, discloses a method for producing cadaverine by culturing a transformed Corynebacterium glutamicum or E. coli engineered to express and secrete both lysine and lysine decarboxylase into the culture medium. In another example, U.S. Pat. No. 11,155,840, incorporated herein by reference in its entirety, discloses a microorganism from a Bacillus sp, such as B. subtilis, licheniformis, engineered to produce lysine and a thermophilic lysine decarboxylase which produces PDA by culturing said Bacillus sp.

In yet other preferred embodiments, the feed stock for the column may be obtained by decarboxylating an amino acid in vitro with a purified or partially purified an amino acid decarboxylase or a cell extract obtained from a culture of a microorganism producing said decarboxylase. For example, U.S. Pat. No. 10,351,839, incorporated herein by reference, discloses a microorganism engineered to produce pH stable lysine decarboxylase.

### Simulated Moving Bed (SMB)

The present invention is best practiced using a simulated moving bed (SMB) system. A SMB system suitable for separating at least a diamine from a feed mixture comprise several column segments forming more than one zone, each of which perform a chromatographic task. For example, a SMB may comprise a first zone of column segments to separate a desired product component, a second zone to separate a second component, and one or more rinse zones to regenerate the resin. Each zone comprises a plurality of segments, each of which contains a bed of the solid strong anion exchange resin. Each zone may further comprise one or more injection points for a feed mixture; one or more injection points for an eluent, the eluent comprising, for example, water or aqueous solution of hydroxide salt; a take-off point for an extract stream; and a take-off point for a raffinate stream. For the most part, columns in each zone are in fluid communication with each other however certain zones may be disconnected from other zones for purposes of applying a wash or regeneration step.

The SMB may be equipped with plurality of valves that are attached to each column segment in a manner such that any feed stream may be introduced to any section or zone, and any outlet or effluent stream may be withdrawn from any section or zone. In certain embodiments, the plurality of valves rotate as rotary unit over stationary column segments stepping from one segment to an adjacent segment in a direction opposite the direction of fluid flow through the SMB system mimicking the effect that would result if the solid phase was moving while the liquid phase was stationary. In other embodiments, the valves may remain stationary, and the column segments are moved as a rotary unit beneath stationary valves in a direction opposite the flow of fluid to provide the same effect.

During operation of the SMB, the inlet connections to which the feed streams are fed and the outlet connections from which the outlet streams are withdrawn are periodically moved, or indexed, from their respective columns to adjacent columns. For example, in one aspect, to achieve separation of at least one diamine from an amino and/or carboxylic acid, the locations of the inlet and outlet streams may be moved intermittently, from column to the next adjacent column, in the opposite direction of liquid eluent flow. The intermittent port movement in the direction of liquid eluent flow simulates the counter-current movement of the bed or beds of the solid adsorbent. Different equipment and operational strategies may be used to simulate the counter-current movement of the solid with respect to the liquid. Any known simulated or actual moving bed chromatography apparatus may be utilized for the purposes of separating of diamines from amino and/or carboxylic acids and salts thereof from a feed mixture such as a fermentation product. As non-limiting examples, apparatuses described in U.S. Pat. Nos. 2,985,589; 3,696,107; 3,706,812; 3,761,533; FR-A-2103302; FR-A-2651148; FR-A-2651149; U.S. Pat. Nos. 6,979,402; 5,069,883; and 4,764,276, each of which is incorporate herein by reference in its entirety, may be configured and operated, according to the present disclosure, for separating of a diamine from amino and/or carboxylic acids and salts thereof as well as inorganic salts such as, but not limited to phosphate and/or sulfate from a feed mixture such as a fermentation product.

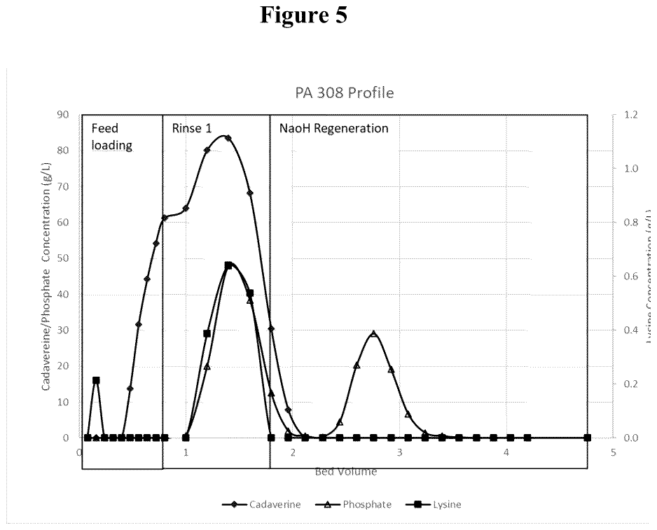

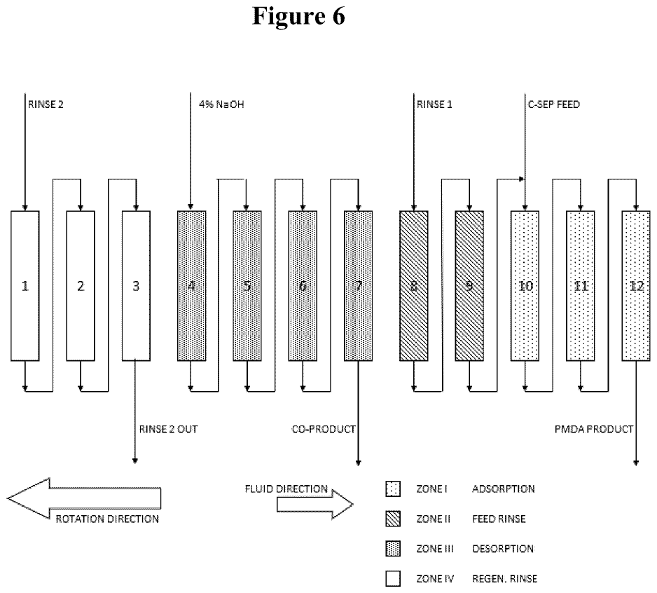

An exemplary process for the separation of diamine from amino and carboxylic acids and salts thereof, a strong anion exchange resin such as but not limited to Mitsubishi PA DIAION PA308™, DOW 22™, and HPA 25L™ is packed into a set of columns contained in a simulated moving bed apparatus configured with a feedstock loading zone, a raffinate elution zone, and a regeneration zone (see for example FIG. 5). The feedstock is loaded onto a first column segment defining the feedstock loading zone. The diamine is eluted from another column segment downstream from the feedstock loading zone relative to the direction of flow of liquid through the column segments. Amino and/or carboxylic acid and the conjugate anion of the salt of the amino and/or carboxylic acid as well as inorganic salts are eluted from yet another column segment in the raffinate elution zone. The column(s) is regenerated in the regeneration zone. In such a configuration, the raffinate elution zone is not in fluid communication with the feedstock loading zone and the regeneration zone is not in fluid communication with the raffinate elution zone. In a preferred embodiment, the water rinse is introduced into the column in a segment upstream of the first column segment defining the feedstock loading zone; a hydroxide salt solution is introduced into the column in a segment within the raffinate elution zone; and water is introduced into a column segment in the regeneration zone (see FIG. 6).

### EXAMPLES

In the examples that follow, the concentration of a lysine refers to the concentration of the lysine calculated as the lysine free base concentration prior to the addition of the anionic acid to form the lysine salt. Unless otherwise stated, in Examples 1-9 the reaction used 10 ml of the lysine salt solution.

### Example 1

### Conversion of 9.7% Lysine Sulfate to 1,5-Pentanediamine (PDA) at pH 5.6

100.52 g of a 9.7 w % lysine sulfate solution (0.66 M lysine) was pH adjusted to 5.6 with sulfuric acid and preheated to 37° C. with 1.80 mg of pyridoxal 5 phosphate in a 100 ml reaction to which 4.0 ml of a cell free crude lysate containing E. coli lysine decarboxylase (CadA) having a protein concentration of about 12 mg/ml was added. The mixture was gently agitated for 24 hours and sampled regularly. The reaction reached 71% lysine conversion to cadaverine within 3 hours with 98% lysine conversion at 24 hours showing the lysine sulfate can be used as suitable substrate for CadA.

### Example 2

### Conversion of 8.4% Lysine Phosphate to PDA at pH 5.5

300.98 g of a 8.4 w % lysine phosphate solution (0.57M lysine) was pH adjusted to 5.5 with phosphoric acid obtaining a final volume of about 290 ml and preheated to 37° C. 146.3 mg of protein was added from a cell free crude lysate containing E. coli lysine decarboxylase and 5.08 mg of pyridoxal 5 phosphate were added. The mixture was gently agitated for 24 hours with samples being pulled throughout the reaction. Final conversion was reached 3 hours with 98% lysine conversion. The results indicate that lysine phosphate can be used as suitable substrate for CadA.

### Example 3

### Conversion of 13.5% Lysine Phosphate to PDA at pH 8.0

100.93 g of 13.5 w % lysine phosphate solution (0.92 M lysine) was adjusted to a pH of 7.958 with phosphoric acid and 49.4 mg of protein were added from a cell free crude lysate with 1.60 mg of pyridoxal 5 phosphate. The mixture was heated to 37° C. and gently agitated for 24 hours. Samples were quenched and 96% conversion by 100 minutes and reached final conversion of 98% lysine was reached within 3 hours. The results indicate that CadA can work at pH 8.0 with high efficiency using lysine phosphate.

### Example 4

### Conversion of 37.1% Lysine Phosphate to PDA at pH 7.7

98.09 g of 37.1 w % lysine phosphate solution (2.54 M lysine) was adjusted to a pH of 7.727 with phosphoric acid obtaining a 98 ml sample to which 150.17 mg of protein were added from a cell free crude lysate with 1.70 mg of pyridoxal 5 phosphate. The mixture heated to 37° C. and was gently agitated for 24 hours. Samples were quenched and final conversion of 70% lysine was reached by 24 hours.

### Example 5

### Conversion of 41.8% Lysine Phosphate to PDA at pH 7.9

109.01 g of 41.8 weight percent lysine phosphate solution (2.86 M lysine) was adjusted to a pH of 7.91 with phosphoric acid and 174.96 mg of protein was added from a cell free crude lysate with 1.70 mg of pyridoxal 5 phosphate. The mixture heated to 37° C. and was gently agitated for 24 hours. Samples were quenched and final conversion of 48% lysine was reached by 24 hours.

### Example 6

### Conversion of 33.6% Lysine Phosphate to PDA at pH 8.4

10 mL of 33.6 w % lysine phosphate solution (2.30 M lysine) was adjusted to a pH of 8.40 with phosphoric acid and with 5.6 mg of pyridoxal 5 phosphate. The feed was split four ways to be dosed with the crude cell extract. Two tubes were dosed with a lower protein amount of 119 mg and 127 mg of protein and two tubes were dosed with a higher protein amount of 256 and 262 mg of protein. The control contained no added enzyme. All samples were incubated at 37° C. The low dosed showed 30.4 and 39.6% lysine conversion in 24 hours. The high dosed samples had 79.5% and 83.4% conversion of lysine in 24 hours.

### Example 7

### Conversion of 17.9% Lysine Phosphate to PDA at pH 8.5

100.57 g of 17.9 w % lysine phosphate solution (1.22 M lysine) was adjusted to a pH of 8.469 with phosphoric acid and 49.84 mg of protein were added from a cell free crude lysate with 1.70 mg of pyridoxal 5 phosphate. The mixture heated to 37° C. and was gently agitated for 24 hours, Samples were quenched and final conversion of 95% within three hours and 97% lysine was reached by 24 hours.

### Example 8

### Conversion of 16.8% Lysine Phosphate to PDA at pH 8.4

A 16.8 w % lysine phosphate solution (1.15 M lysine) was made to which 2 liters of a crude CadA extract was added (about 13.8 g protein) in a final reaction volume of 29072.67 mL with 507.1 mg of pyridoxal 5 phosphate. The final pH was 8.39. The reaction was gently agitated at 37° C. and samples were taken periodically. 95% conversion was obtained in the first hour and 99.5% conversion was achieved by 24 hours.

### Example 9

### Conversion of 18.9% Lysine Phosphate to PDA at pH 9.0

100.58 g of 18.9 w % lysine phosphate solution (1.29 M lysine) was adjusted to a pH of 8.949 with phosphoric acid and 49.84 mg of protein was added from a cell free crude lysate with 1.70 mg of pyridoxal 5 phosphate and heated to 37° C. The mixture was gently agitated for 24 hours and samples were quenched and final conversion of 63.5% within three hours and 85.2% lysine was reached by 24 hours.

### Example 10

Separation of 1,5-Pentanediamine from a Feed Stock:

### (a) Feed Stock:

The feedstock is a mixture resulting from the decarboxylation of lysine by the action of lysine decarboxylase after forming lysine phosphate by adding sufficient phosphoric acid to lysine base to adjust the pH to 8.5 as described in Example 8. The feed stock contained 90 g/L 1,5-pentanediamine, 0.27 g/L lysine and 40 g/L phosphate.

### (b) Preparation of Strong Anion Exchange Columns:

A slurry of each Dowex22™, PA308™, and HPA25L™ strong anion exchange resins (100 mL) in deionized water was load into a jacketed glass column. Air bubbles were removed from the resin bed, and the bed was rinsed with three bed volumes of degassed deionized water at a flow rate of three bed volumes/hour. The packed column was conditioned by pumping five bed volume of 4% sodium hydroxide solution at a flow rate of five bed volume/hour, followed by pumping degassed deionized water at the same flow rate, until effluent pH stabilized. The liquid level in the column is lowered until even with the top of the resin bed.

### (c) Purification of 1,5-Pentanediamine:

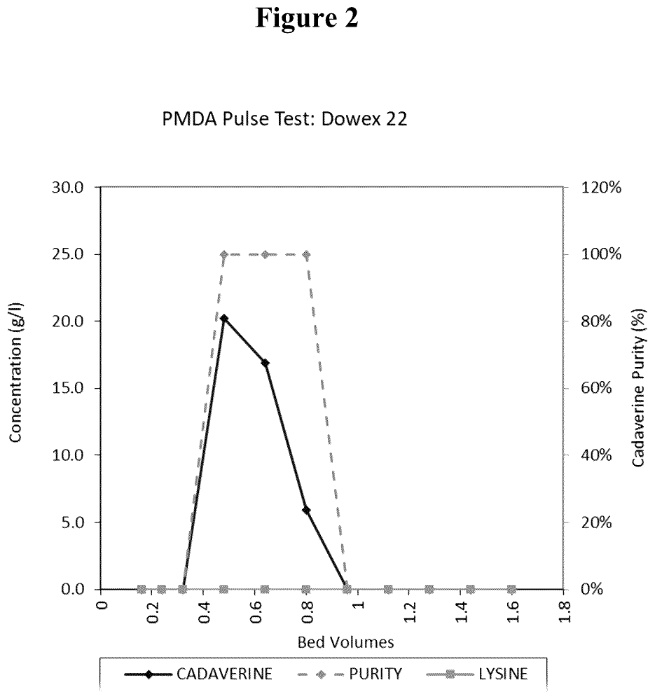

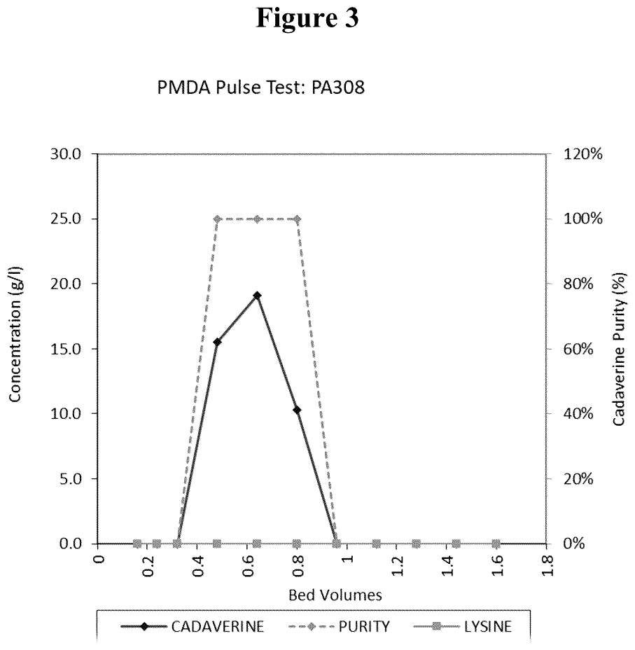

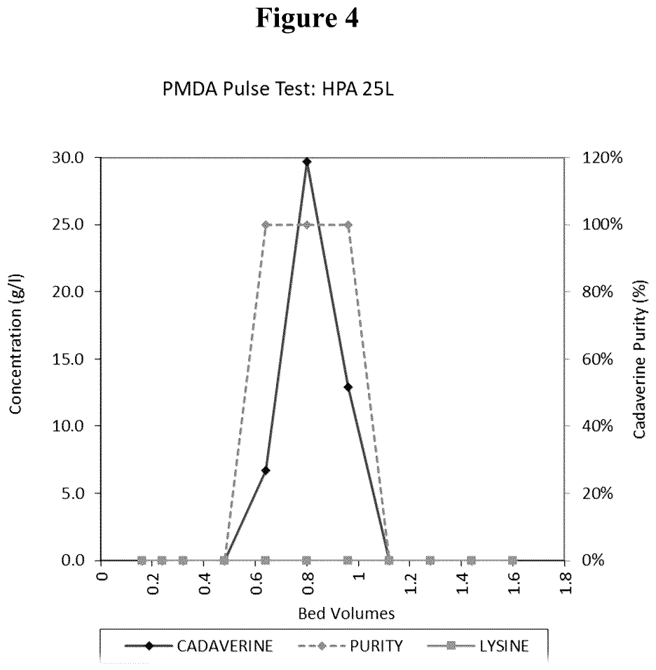

About 0.8-1.0 bed volume of the feed stock was loaded on the three columns and 1,5-pentanediamine was eluted from the columns at 40° C. at a flow rate of 5 mL/min with degassed and deionized water followed by eluting lysine and phosphate salt by 4% (w/w) aqueous sodium hydroxide solution. Fraction1 of 8 mL (0.08 bed volume) were collected and analyzed for content to determine the purity of 1,5-pentanediamine in each fraction. FIG. 3A shows test comparison of eluting 1,5-pentanediamine (cadaverine) from the anion exchange columns packed with DOW 22, PA208 and HPA 25. FIGS. 2-4 shows the elution profile and purity of 1,5-pentanediamine as well as the lysine content in each fraction. As shown in FIGS. 2-4, the eluted diamine fractions from the columns were >99% pure.

### Example 11

### Effect of the pH on Recovery of 1,5-Pentanediamine:

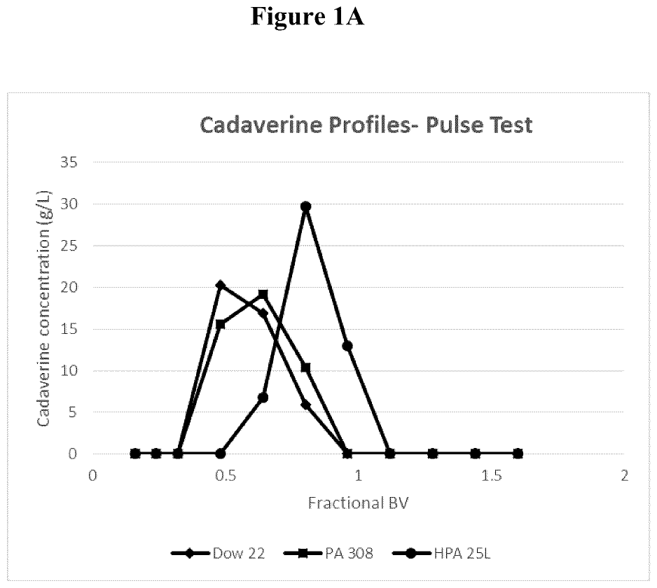

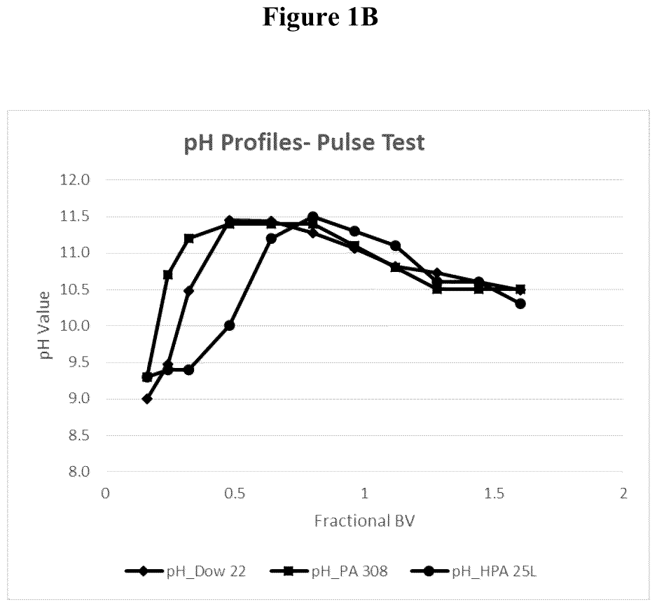

For testing the effect of pH on the elution profile and yield, sodium hydroxide and sulfuric acid were used to adjust the pH of the feed to the desired pH value. FIG. 1B shows the effect of the pH of the feed stock on the elution profile of 1,5-pentanediamine from three columns packed with DOW22, PA308, and HPA25L.

### Example 12

### Single Column Purification of 1,5-Pentanediamine Over PA 308 Resin

The feed was a lysine enzymatic decarboxylation reaction mixture adjusted with phosphoric acid to a pH of 8.5 as described in Example 8 containing 90 g/L PDA, 0.27 g/L lysine, and 40 g/L phosphate. 0.8 bed volumes (BV) of the feed was applied over a 100 ml column (15 mm×600 mm) and then eluted with water at two different feed rates then with 4% NaOH at the feed rates shown below.
Fraction Collecting: Fractions were collected every (8 mLs.) 0.08 BV
Column Temperature: 40° C.
Feed Rate (0.0-0.8 BV): 32 mLs/min
Elution 1 Rate (0.8-1.8 BV): 32 mLs/min
Elution 2 Rate (1.8-4.8 BV): 5 mLs/min
Eluent 1 (0.8-1.8 BV): Degassed DI Water
Eluent 2 (1.8-4.8 BV): 4% NaOH

As can be seen in FIG. 5, the PDA (cadaverine) product was collected in the initial feed loading and water rinse (Rinse 1) fractions at high purity after ˜0.3 1.0 BVs after which time the lysine and phosphate were seen in the effluent. This was followed by regeneration of the column by 4 elution with % NaOH, removing the adsorbed phosphate and residual cadaverine and lysine. The cadaverine collected in the product fraction up to 1 BV was 100% purity.

### Example 13

### Purification of 1,5-Pentanediamine Using Simulated Moving Bed:

FIG. 6 shows a schematic representation of the simulated moving bed chromatographic system designed for the purification of diamines from a complex reaction mixture. Each of the 12 column segments of 450 ml were packed with the strong base anion exchange resin PA 308. A feed sample was obtained as described in Example 12 having the composition shown in Table 2 below and loaded unto the resin continuously at 1.15 liters/hour. It was loaded on column 10 in the adsorption zone I and 1,5-pentanediamine product was continuously collected from column 12. In zone II, the columns were continuously rinsed with water. In zone III, the desorption zone, the negatively charged ions and residual lysine were continuously eluted from the columns with 4% sodium hydroxide solution. Samples of the product diamine, the eluate from desorption zone, the first (raffinate) and rinse 2 (sodium hydroxide eluate) were collected and analyzed, and their composition is listed in Table 2.

TABLE 2

| | 1,5- | | | | |
|---|---|---|---|---|---|
| | pentanediamine | Lysine | Phosphate | Sulfate | Diamine |
| Sample | g/L | g/L | mg/L | mg/L | Purity % |
| Feed | 95.25 | 4.3 | 39244 | 2945 | 68.6 |
| Product | 20.0 | 0.0 | 1 | 0 | 100% |
| Raffinate | 3.1 | 1.7 | 9366 | 299 | |
| Rinse 2 | 0.5 | 0 | 43 | 482 | |

1. A process for separating a diamine from an aqueous feed stock containing the diamine, and at least one conjugate anion salt of an amino acid or carboxylic acid, comprising: contacting the aqueous feed stock at a pH in the range 5.0 to 10.0 with a bed of a strong anion exchange resin, and eluting the diamine from the resin in a fraction that has a purity of at least 85% and contains less than 10% (w/w) of the amino acid or carboxylic acid and conjugate anion.

2. The process of claim 1, wherein the diamine is 1,5-pentanediamine (PDA) and the at least one amino acid salt is a lysine salt.

3. The process of claim 1, wherein the feed stock is obtained by decarboxylation of lysine by the action of lysine decarboxylase.

4. The process of claim 3, wherein the lysine salt is selected from the group consisting of lysine phosphate and lysine sulfate.

5. The process of claim 4, wherein less than 10% of the sulfate or phosphate conjugate anion of the lysine salt is present in the eluted fraction containing the diamine.

6. The process of claim 5, wherein the lysine salt is lysine phosphate.

7. The process of claim 2, wherein the strong anion exchange resin comprises a quaternary ammonium salt.

8. The process of claim 2, wherein the purity of PDA in the eluted fraction is at least 95% and contains less than 5% (w/v) lysine.

9. The process of claim 1, wherein at least one amino and/or carboxylic acid is eluted from the anion exchange bed with a hydroxide salt.

10. The process of claim 2, wherein the lysine is eluted from the anion exchange bed with a hydroxide salt.

11. The process of claim 10, wherein the hydroxide salt is ammonium hydroxide.

12. The process of claim 10, wherein the hydroxide solution is sodium hydroxide.

13. The process of claim 1, wherein the feed stock has a pH in the range of 8.0 to 9.0.

14. The process of claim 2, wherein the bed is packed into a set of columns contained in a simulated moving bed apparatus configured with a feedstock loading zone, a raffinate elution zone and a regeneration zone, wherein: (a) the feedstock is loaded onto a first column segment defining the feedstock loading zone, (b) the 1,5-pentanediamine is eluted from a second column segment downstream from the feedstock loading zone relative to the direction of flow of liquid through the column segments, (c) lysine and the conjugate anion of the salt of the lysine are eluted from a third column segment in the raffinate elution zone, and (d) the column is regenerated in the regeneration zone; and where the raffinate elution zone is not in fluid communication with the feedstock loading zone and the regeneration zone is not in fluid communication with the raffinate elution zone.

15. The process of claim 14, wherein a water rinse is introduced into the column in a segment upstream of the first column segment defining the feedstock loading zone; a hydroxide salt solution is introduced into the column in a segment within the raffinate elution zone; and water is introduced into a column segment in the regeneration zone.

16. The process of claim 14, wherein the lysine salt is lysine phosphate.

17. The process of claim 14, wherein the simulated moving bed apparatus comprises 12 column segments of which, the feedstock loading zone comprises 5 column segments, the raffinate elution zone comprises 3 column segments and the regeneration zone comprises 4 column segments.

18. The process of claim 17, wherein the lysine salt is lysine phosphate.

19. A method of producing 1,5-pentanediamine (PDA) comprising:
(a) obtaining a culture broth resulted from fermenting a host microorganism engineered to produce lysine;
(b) removing the microorganism from the culture broth;
(c) adding a source of phosphate or sulfate to the culture broth to form a phosphate or sulfate salt of lysine;
(d) decarboxylating the lysine by contacting culture broth containing the sulfate of phosphate with lysine decarboxylase forming a culture broth containing the lysine salt and PDA;
(e) contacting the culture broth containing the lysine salt and PDA with a bed of a strong anion exchange resin in a column, and
(f) eluting the PDA from the column in a first fraction separate from a second fraction containing lysine and lysine salt;
wherein the PDA is eluted from the column with at least 95% purity and contains less than 5% lysine.

20. The method of claim 19, wherein the column comprises a set of columns contained in a simulated moving bed apparatus configured with a feedstock loading zone, a raffinate elution zone and a regeneration zone, wherein: (a) the feedstock is loaded onto a first column segment defining the feedstock loading zone, (b) the 1,5-pentanediamine is eluted from a second column segment downstream from the feedstock loading zone relative to the direction of flow of liquid through the column segments, (c) lysine and the conjugate anion of the salt of the lysine are eluted from a third column segment in the raffinate elution zone, and (d) the column is regenerated in the regeneration zone; and where the raffinate elution zone is not in fluid communication with the feedstock loading zone and the regeneration zone is not in fluid communication with the raffinate elution zone.

21. A method of forming 1,5-pentanediamine (PDA) comprising contacting a mixture containing at least a 0.3M solution of a lysine salt with sulfate or phosphate as the conjugate anion with an enzyme having a lysine decarboxylase activity for a time sufficient to convert at least 50% of the lysine to PDA.

22. The method of claim 21 wherein the mixture is at a pH of 8.0 to 9.0.

23. The method of claim 21 wherein the mixture contains 0.5 to 2.9 M lysine phosphate at a pH of 8.0 to 9.0.

24. The method of claim 23 wherein the pH is 8.0 to 8.5.

25. The method of claim 21 wherein at least 90% of the lysine is converted to PDA.

26. The method of claim 21 wherein the lysine decarboxylase is encoded by the cadA gene of E. coli.

27. The method of claim 21, further comprising separating the PDA from lysine and the conjugate anion by contacting the reaction mixture with a strong base ion exchange resin and eluting the PDA from the resin, wherein the eluted PDA is at least 85% pure containing less than 15% of the lysine or conjugate anion.

28. The method of claim 27 wherein the strong base ion exchange resin is configured in a simulated moving bed apparatus.

29. The method of claim 28 wherein the simulated moving apparatus comprises a set of columns configured with a feedstock loading zone, a raffinate elution zone and a regeneration zone, wherein: (a) the feedstock is loaded onto a first column segment defining the feedstock loading zone, (b) the 1,5-pentanediamine is eluted from a second column segment downstream from the feedstock loading zone relative to the direction of flow of liquid through the column segments, (c) lysine and the conjugate anion of the salt of lysine are eluted from a third column segment in the raffinate elution zone, and (d) the column is regenerated in the regeneration zone; and where the raffinate elution zone is not in fluid communication with the feedstock loading zone and the regeneration zone is not in fluid communication with the raffinate elution zone.

30. The process of claim 29, wherein a water rinse is introduced into the column in a segment upstream of the first column segment defining the feedstock loading zone; a hydroxide salt solution is introduced into the column in a segment within the raffinate elution zone; and water is introduced into a column segment in the regeneration zone.

31. The process of claim 29, wherein the lysine salt is lysine phosphate.

32. The process of claim 29, wherein the simulated moving bed apparatus comprises 12 column segments of which, the feedstock loading zone comprises 5 column segments, the raffinate elution zone comprises 3 column segments and the regeneration zone comprises 4 column segments.
